(19) United States
(12) Patent Application Publication
(10) Pub. No.: US 2025/0090736 A1
(43) Pub. Date: Mar. 20, 2025

(54) Transfer Set with Integral Access Port

(71) Applicant: Light Line Medical, Inc., Salt Lake City, UT (US)

(72) Inventors: Lynn E. Jensen, Syracuse, UT (US); Curtis D. Long, Salt Lake City, UT (US)

(73) Assignee: Light Line Medical, Inc., Salt Lake City, UT (US)

(21) Appl. No.: 18/470,753

(22) Filed: Sep. 20, 2023

(51) Int. Cl. A61M 1/28; A61L 2/08; A61L 2/26; A61M 39/10; A61M 39/16; A61M 39/20; A61M 39/28

(52) U.S. Cl. CPC A61M 1/285; A61L 2/08; A61L 2/26; A61M 39/1011; A61M 39/105; A61M 39/16; A61M 39/20; A61M 39/28; A61M 39/288; A61L 2202/11; A61L 2202/24; A61M 2039/167; A61M 2207/00; A61M 2209/06

(57) ABSTRACT

A multifunctional transfer set having an access port is disclosed. The multifunctional transfer set facilitates the introduction of elongate members insertably/retractably and allows fluid flow therethrough without requiring detachment during use. The multifunctional transfer set has a transfer set body assembly connected to a Y-adapter with an access port and a longitudinal portion. A longitudinal axis extends through the transfer set body assembly and longitudinal portion of the Y-adapter and not through the access port. An extension sub-assembly is connected to the access port, the extension sub-assembly having branch tubing, a flow-control device, and a coupling assembly allows fluid flow therethrough into and out of the transfer set body assembly if the flow-control device is open and prevents fluid from flowing therethrough if the flow-control device is closed. An existing transfer set without intervening side access may be converted into a multifunctional transfer set with a shorter longitudinal length.

### BACKGROUND OF THE INVENTION

### 1. Field of the Invention

The present invention relates to transfer sets with integrated access port for use by clinicians and patients because the integrated access port may be used to inject medications and/or extract sample fluids from a patient, eliminating the need to connect other devices to the transfer set to do these and other actions. More specifically, the present invention relates to a peritoneal dialysis transfer set with integrated access port wherein the integrated access port may be used for placing a disinfecting fiber within the transfer set.

### 2. The Relevant Technology

Peritoneal dialysis (PD) is a treatment for kidney failure that uses the peritoneum in a person's abdomen as the membrane through which fluid and dissolved substances are exchanged with the patient's blood. Peritoneal dialysis removes excess fluid, corrects electrolyte problems, and removes toxins in patients with kidney failure. Peritoneal dialysis has better outcomes than hemodialysis during the first couple of years. Other benefits include greater flexibility, lifestyle advantages (such as home treatments and better patient mobility) and better tolerability in those patients with significant heart disease. Additionally, peritoneal dialysis is significantly less expensive than hemodialysis, about $53,000 per year compared to about $72,000 per year according to information from the U.S. Renal Data System.

Typically, a few weeks before the patient starts peritoneal dialysis, a surgeon places a permanent, soft tube catheter into the patient's abdomen. In peritoneal dialysis, a specific solution, known as a dialysate, is introduced through the permanent, indwelling peritoneal catheter in the lower abdominal cavity and when the dialysis cycle is completed the dialysate delivery is discontinued and the indwelling peritoneal catheter capped off so that the patient may move freely about. The dialysis cycle may cither occur at regular intervals throughout the day, known as continuous ambulatory peritoneal dialysis (CAPD), or at night with the assistance of a machine, known as automated peritoneal dialysis (APD). The solution is typically made of sodium chloride, bicarbonate, and an osmotic agent such as glucose.

Peritonitis is a serious infection of the peritoneum, the tissue that lines the abdomen and protects internal organs. It can be caused by a hole in your digestive tract, a burst appendix, or an infection of the fluid that accumulates in some people with liver or kidney failure. Peritonitis can cause severe pain, fever, nausea, vomiting, and bloating. It can also lead to sepsis, organ failure, and death if not treated quickly. Peritonitis is a serious risk to peritoneal dialysis patients because a common cause of peritonitis is touch contamination, e.g. insertion or handling of the peritoneal dialysis catheter (also referred to as a PD catheter) or connecting/disconnecting the dialysate delivery mechanism to/from the indwelling PD catheter by un-sanitized hands, which potentially introduces bacteria to the abdomen.

Best practice disinfecting procedures have been adopted to reduce the incidence of peritonitis for peritoneal dialysis patients, but peritonitis remains a serious concern for clinical and especially at-home peritoneal dialysis patients. Various types of disinfection techniques have been used with limited success.

However, one very promising disinfecting technique is the delivery of non-ultraviolet light inside catheters and/or extension sets to prevent, reduce, or eliminate infectious agents before, during, or after a medical procedure. See U.S. Pat. Nos. 11,229,728; 11,229,808; and 11,497,932 that disclose light delivery techniques and options both inside and outside the body providing effective sterilizing that enhances prevention, reduction, and elimination of infectious agents throughout the dialysis system, including in, on, or around the catheter, in, on, or around catheter extensions or connectors, and/or on or in tissue surrounding the catheter while in a body cavity.

Present PD extension sets are used as a portion of the dialysate delivery mechanism that connects to the indwelling PD catheter and such PD extension sets have been used for years. See, for example, U.S. Pat. Nos. 5,533,996; 5,582,600; 8,377,012; and 8,636,706 that disclose PD extension sets (including extensions known as transfer sets) that are to be disposed between the PD catheter and the fresh and waste dialysate reservoirs. However, such PD extension sets do not provide access for delivery of disinfecting light directly into the extension set or the PD catheter through the extension set.

### SUMMARY OF THE INVENTION

The exemplary embodiments of this disclosure relate to transfer sets with an integral access port for introducing and inserting elongate members such as, for example, a guide wire, an endoscope, an angioscope, a hysteroscope, a gastroscope, a flexible telescope for colonoscopies, bronchoscope, a cystoscope, probes for illumination, probes for pinpoint medication or treatment delivery, probes for inflation gas delivery, fiber optics, and any of numerous other devices or instruments that are elongate and may be inserted into and/or retracted from a cavity of a patient's body and/or from within tubular structures outside a patient's body.

Frequently, transfer sets are used with catheters and other tubular structures to facilitate delivery of fluids from outside the body to inside the body and retrieval of fluids from inside the body to outside the body, and reducing, inhibiting, preventing, or eliminating infectious agents before, during, or after delivery and/or retrieval of fluids may be of paramount concern. Transfer sets with an access port eliminate the need to disconnect an already established delivery/retrieval channel for fluid flow when any of the above-mentioned elongate members are to be introduced and inserted into the delivery/retrieval channel.

There is a great need for increasing the efficacy of in-home and clinical peritoneal dialysis treatments for dialysis patients. Peritoneal dialysis is already considerably less expensive than hemodialysis, but more effective disinfection will drive the overall costs of peritoneal dialysis even lower, making peritoneal dialysis more attractive to both in-home and clinical use. Peritoneal dialysis already offers patients better lifestyle choices. In-home treatment, in the comfort and privacy of a patient's own home, is better than clinical treatment, and clinical treatment is better than hospitalization for most patients. Additionally, by increasing effective and safe home-conducted peritoneal dialysis, the stress and strain is less taxing on health institutions dealing with dialysis. Also, because in-home peritoneal dialysis is less expensive it will be more likely for a patient's insurance to cover in-home treatment.

Best practice disinfecting procedures are doing their part in reducing the rate of infection incidents, but such practices address only prevention of infection and some reduction of infections that may be presented where the disinfecting procedures are applied. Best practice disinfecting procedures, though helpful and needed, are severely limited. Once an infection is introduced or presents itself, if unchecked, migration of the infection within the dialysis system may lead to costly and painful changing out the catheter, or to serious infection within the patient's body, of which peritonitis is of paramount concern.

Hence, an introducing assembly or system that enables the appropriate elongate member to be introduced into catheters or other tubular structures, without compromising sterility or unduly inhibiting fluid flow, would advance in-home as well as institutional medical treatments. This is particularly true with in-home peritoneal dialysis.

Some exemplary embodiments of this disclosure are directed to a kit including an introducer assembly for introducing an elongate member into a tubular-receiving structure, where the introducer assembly has a ready mode, an introduced mode, and a detached mode. The ready mode being a configuration of the introducer assembly during shipping and/or storage typically wherein the introducer assembly is maintained sterile, and the elongate member is housed and secured within a protective guide tube. The introduced mode being a configuration of the introducer assembly after being connected to the tubular-receiving structure and the elongate member is advanced into the tubular-receiving structure to reside therein after securing the elongate member in place. Once the elongate member is secured in place, the guide tube of the introducer assembly may be detached from the elongate member for disposal, thereby changing the introducer assembly from the introduced mode to detached mode.

By making at-home administered peritoneal dialysis safer, lifestyle-friendly, and more cost-effective, that option for eligible dialysis patients becomes much more attractive, not only to patient's, but to caregivers, healthcare providers, and insurance companies as well. Furthermore, any increase in home-conducted peritoneal dialysis reduces dialysis stress on the health institutions dealing with dialysis.

By packaging the multifunctional transfer set a pre-packaged kit along with an elongate member, contents of the pre-packaged kit may be sterilized and compatibly matched in size and structure to operate together with the elongate member to accomplish a designed purpose. Further, the elongate member being selected from the group of elongate members including a guide wire, an endoscope, an angioscope, a hysteroscope, a gastroscope, a flexible telescope for colonoscopies, bronchoscope, a cystoscope, probes for illumination, probes for pinpoint medication or treatment delivery, probes for inflation gas delivery, and fiber optics. Moreover, dimensions of the multifunctional transfer set may be compatibly matched the elongate member selected so that, for example, a maximum diameter of the elongate member is less than a minimum diameter of the multifunctional transfer set.

Also, when the contents of the pre-packaged kit include a fiber optic, an introducer assembly, disposed in a ready mode, and having a length to receive and hold the fiber optic in position for introduction into the multifunctional transfer set may be included. If a fluid extension line for connection to the multifunctional transfer set is also included, the fiber optic may be pre-selected to have a length to extend through the multifunctional transfer set and into and/or through the fluid extension line.

The multifunctional transfer sets disclosed herein are ideal companions for pre-packaging into kits for distribution, complementary use of pre-selected compatibly matched component parts and fittings, and facilitating the implementation the multifunctional transfer sets into the delivery of medical services and treatments, enhancing overall performance, improving efficiency and efficaciousness, reducing costs, minimizing errors, preventing/reducing/eliminating infections, and simplifying assembly of the compatibly matched companion parts.

### BRIEF DESCRIPTION OF DRAWINGS

For the above-recited and other features and advantages of the invention to be readily understood, a more particular description of the invention briefly described above will be rendered by reference to specific embodiments thereof which are depicted or illustrated in the appended figures. Understanding that these depictions and drawings show only typical embodiments of the invention and should not be considered limiting of its scope, the invention will be described and explained with additional specificity and detail with reference to the accompanying figures in which:

FIG. 1 is a side elevational view of a representative prior art transfer set showing a transfer set hub assembly, intermediate tubing and a luer connector assembly.

FIG. 1A is a side elevational view of a transfer set hub isolated from the transfer set hub assembly of the representative prior art transfer set of FIG. 1, the transfer set hub having a pair of tines and at least one bump stop.

FIG. 2 is a side elevational view of an exemplary transfer set of the present invention having a transfer set body assembly, a Y-adapter having a longitudinal portion and an integral access port, a vented cap, branch tubing, and a luer connector assembly.

FIG. 2A is a directionally reversed side elevational view of an alternative exemplary transfer set with an alternative transfer set body assembly and wherein the vented cap and a protective cap removed to reveal a male luer and luer connector.

FIG. 3 is a side elevational view of an exemplary transfer set body isolated from the exemplary transfer sets of FIGS. 2 and 2A. Note that FIG. 3 also depicts the transfer set hub of FIG. 1A with the pair of tines and any bump stops removed.

FIGS. 4A-C is a series of side elevational views showing an exemplary body sub-assembly comprising a combination of the luer connector, the transfer set body, and a coupler tubing. FIG. 4A depicts the transfer set body connected to luer connector. FIG. 4B depicts the coupler tubing connected to the luer connector, the transfer set body having been removed so not to obscure the coupler tubing. FIG. 4C is a partial vertical cross-section view of the connection of the coupler tubing to the luer connector showing a hollow flange portion of the luer connector disposed within a tubing lumen of the coupler tubing.

FIGS. 5A-C is a series of side elevational views showing an exemplary Y-adapter having a longitudinal portion and an integral access port. FIG. 5A depicts the integral access port of Y-adapter as a barbed side port directed to the right. FIG. 5B depicts the Y-adapter rotated 90° so that the barbed side port is directed forward. FIG. 5C depicts a sectional view along Section C-C of FIG. 5B to show the inner profile of the Y-adapter.

FIG. 6 is a side elevation view of an exemplary extension tubing sub-assembly comprising extension tubing, a pinch clamp, a barbed connector, a tubing overlay, and a ring cap securing a luer (not visible).

FIG. 7 is a side elevation view of an exemplary combination of the extension tubing sub-assembly connected to the Y-adapter and showing a compression sleeve in sectional cut-away.

FIG. 8 is a perspective view of an exemplary transfer set with integral access port connected to a light engine via a light transmission cord, a dialysis bag assembly via an extension set, and a peritoneal catheter showing an exemplary set up using a transfer set with integral access port to facilitate light delivery to disinfect/sterilize the extension set.

FIG. 9 is a perspective view of an exemplary transfer set with integral access port connected to a light engine via a light transmission cord, a dialysis bag assembly via an extension set, and a peritoneal catheter showing another exemplary set up using a dual light delivery configuration for disinfection/sterilization in the extension set and/or the peritoneal catheter.

FIG. 10 is a top plan view of an exemplary light fiber introducer to be connected to the transfer set with integral access port for introduction of the light fiber into and through the transfer set with integral access port.

FIG. 11 is a perspective view of an exemplary transfer set with integral access port showing the light fiber as introduced into and through the transfer set with integral access port while the light fiber introducer is being withdrawn after introduction.

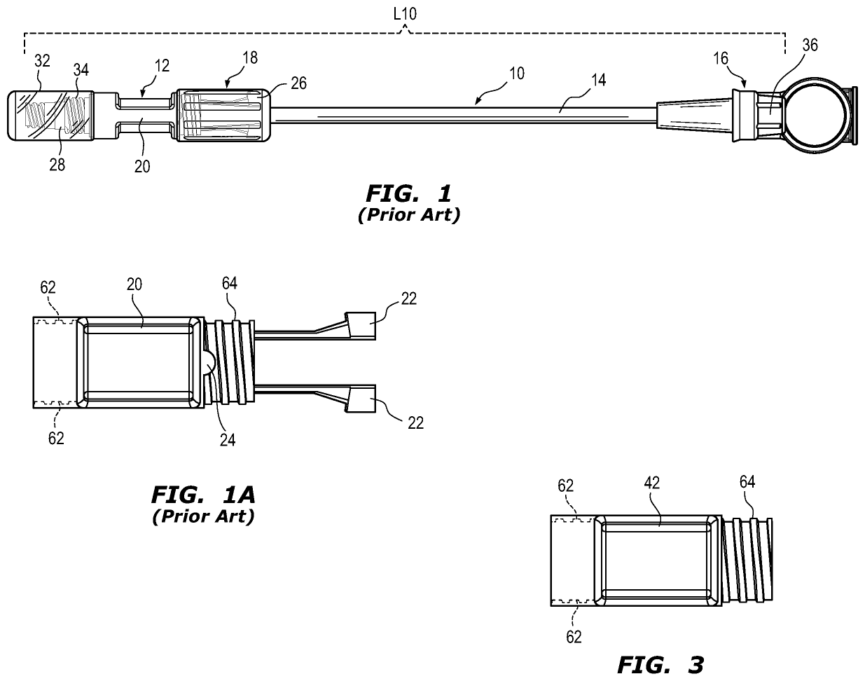

REFERENCE NUMBERS

| | |
|---|---|
| prior art transfer set 10 | transfer set hub assembly 12 |
| intermediate tubing 14 | coupling assembly 16 |
| twisting open/close mechanism 18 | hub body 20 |
| tines 22 (see FIG. 1A) | bump stop 24 |
| twist clamp 26 | core male luer 28 |
| hollow flange 30 | protective cap 32 |
| cap threads 34 | ring cap 36 |
| transfer set 40 | transfer set body 42 |
| access port 44 | Y-adapter 46 |
| vented cap 48 | luer connector 50 |
| male luer 52 | transfer set cap 54 |
| coupler tubing 56 | branch tubing 58 |
| pinch clamp 60 | female threads (transfer set body) 62 |
| male threads (transfer set body) 64 | passageway 66 |
| female threads (transfer set cap) 68 | side port 70 |
| barbed end 72 | annular recess 74 |
| annular spacer 76 | central hollow protrusion 78 |
| shoulder 80 | extension sub-assembly 82 |
| lumen 84 | compression sleeve 86 |
| barbed connector 88 | abutment shoulder 90 |
| inward male threads 92 | disabled twist clamp 94 |
| intermediate connector 96 | peritoneal dialysis catheter (PD |
| | catheter) 98 |
| peritoneal dialysis system 100 | fluid extension line 102 |
| dialysate exchange switch 104 | dialysate supply bag 106 |
| waste dialysate retrieval bag 108 | external coupling end 110 |
| extension line portal 112 | dialysate inlet 114 |
| waste dialysate outlet 116 | exchange selector 118 |
| dialysate 120 (also dialysis solution | feed line 122 |
| 120) | |
| EMR conduction system 124 | light engine 126 |
| light transmission cord 128 | light delivery element 130 |
| connector assembly (proximal) 132 | fiber optic 134 |
| introducer assembly 136 | Tenckhoff coil 138 |
| peritoneal cavity 140 | patient 142 |
| peritoneal lining (membrane or | waste drain line 146 |
| lining) 144 | |
| Y-adapter 148 | light coupling port 150 |
| guide tube 152 | elongate member assembly 154 |
| proximal body 156 | distal portion 158 |
| longitudinal axis 160 | longitudinal slide slot 162 |
| stop 164 | slide collar 166 |
| grip 168 | slide post 170 |
| distal connector assembly 172 | SMA fiber connector 174 |
| longitudinal length L10 | longitudinal length L40 |
| rotation arrows RA | |

### DETAILED DESCRIPTION OF THE INVENTION

Preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the present invention, as represented in the Figure(s), is not intended to limit the scope of the invention, as claimed, but is merely representative of presently preferred embodiments of the invention.

The word “exemplary” is used exclusively herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

A representative prior art transfer set 10 is depicted in FIG. 1. The prior art transfer set 10 depicted is used during peritoneal dialysis and comprises a transfer set hub assembly 12, intermediate tubing 14 and a coupling assembly 16. The transfer set hub assembly 12 comprises a twisting open/close mechanism 18 that controls the flow of fluids through the transfer set 10. This twisting open/close mechanism 18 comprises a hollow hub body 20 with tines 22 (see FIG. 1A) and at least one bump stop 24 (see FIG. 1A), and a twist clamp 26 that engages the hub body 20 in rotational engagement, wherein twisting (rotating) the twist clamp 26 one direction causes tines 22 of hub body 20 to clamp down upon the intermediate tubing 14, pinching it closed. Hence, twisting (rotating) the twist clamp 26 one direction closes the prior art transfer set 10 so that fluids are prevented from flowing through the intermediate tubing 14 of prior art transfer set 10. Twisting (rotating) the twist clamp 26 the opposite direction opens the prior art transfer set 10 so that fluids may freely flow through a longitudinal pathway within the prior art transfer set 10.

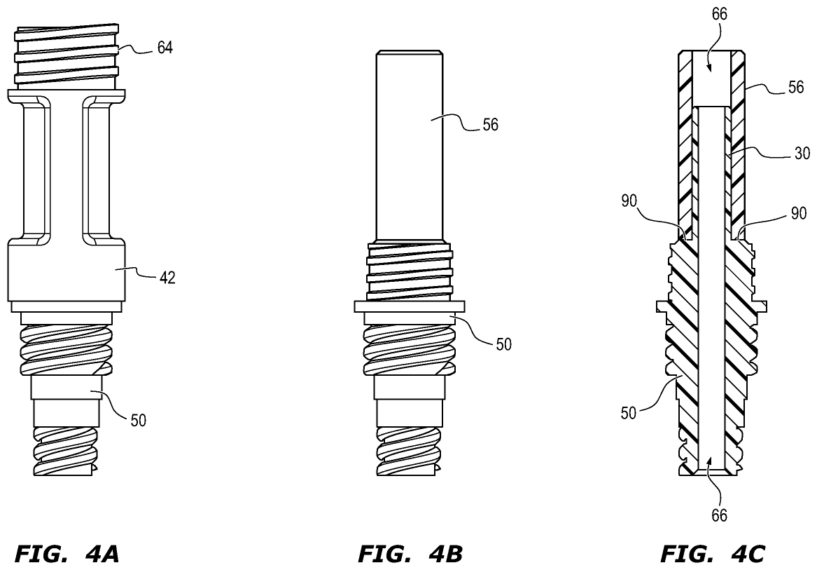

The transfer set hub assembly 12 further comprises a core male luer 28 (partially shown in FIG. 1, see FIG. 4B) with a hollow flange 30 (not visible in FIG. 1, see FIG. 4C) that connects to the intermediate tubing 14. A portion of the core male luer 28, including a hollow flange 30 and a connected end of the intermediate tubing 14 nests within the hollow of the hub body 20. The core male luer 28 being threadedly secured within the hollow of the hub body 20. The visible portion of the core male luer 28 is shown through and is being protected by a transparent protective cap 32. The protective cap 32, whether transparent or not, protects and maintains the sterility of the core male luer 28 while in place. In the embodiment shown, the protective cap 32 and the core male luer 28 have mating cap threads 34 that engage to secure the protective cap 32 to the core male luer 28 of transfer set hub assembly 12.

The intermediate tubing 14 is connected and permanently secured between the transfer set hub assembly 12 and the coupling assembly 16, and has a length that, typically, is the variable that determines the length of the of the transfer set 10. Transfer sets are manufactured in several lengths to accommodate various uses. Transfer sets that have been in use in peritoneal dialysis for more than a decade are typically 9-12 inches long.

The coupling assembly 16 has a luer (not visible in FIG. 1) that may have either male threads or female threads or any other suitable luer means for connecting the transfer set 10 to the catheter. The coupling assembly 16 usually also comprises a ring cap 36 to protect and maintain the sterility of the luer threads or other luer connection means. Use of a ring cap 36 facilitates the single-hand detachment/reattachment of the ring cap 36 from the transfer set 10.

The dialysis-related prior art transfer set 10 is designed to connect a dialysis catheter (not shown) to the dialysis bag assembly containing dialysis solution (either fresh or waste, not shown). As mentioned above, a few weeks before a patient starts peritoneal dialysis, a surgeon places the distal end of a permanent, soft tube catheter (one of several types of peritoneal dialysis catheters) into the patient's abdominal cavity so that a portion of the catheter (the proximal end) extends outside of the patient's body. The proximal end of the catheter is secured with a cap that prevents infection. Typically, the luer (not visible in FIG. 1) of coupling assembly 16 is connected to the proximal end of the peritoneal catheter, either directly or indirectly via an extension line, after the cap and the ring cap 36 are removed. This manual coupling of the transfer set 10 to the peritoneal catheter is particularly vulnerable to contamination if not conducted precisely under disinfection protocols and best practices.

In peritoneal dialysis, the solution known as dialysate is introduced through the permanent, indwelling peritoneal catheter into the lower abdominal cavity, and when the dialysis cycle is completed, the delivery of fresh dialysate is discontinued, and the indwelling peritoneal catheter capped off so that the patient may move freely about. The dwell time of the dialysate within the lower abdominal cavity is about 4 to 6 hours, and the waste dialysate is drained by gravity or by machine (a cycler). The cycle of one introduction of fresh dialysate and draining waste dialysate is called an exchange. Most patients need 3 to 4 exchanges each day which take about 30 to 40 minutes each. Consequently, even with careful handling of the transfer set and various connections, infectious agents may be introduced into the dialysis setting, particularly when it is performed at home.

The most frequent and important complication of peritoneal dialysis catheters is infection, which may result in catheter loss and discontinuation of peritoneal dialysis. Infection of the abdominal lining (peritonitis) is a common complication of peritoneal dialysis and can be quite serious. Studies have consistently shown that peritoneal dialysis patients who experience peritonitis were 2-6 times more likely to experience premature mortality within the first 1-2 months post-occurrence of peritonitis, with cardiac mortality being the predominant cause of mortality.

Consequently, preventing, reducing, or eliminating infection in the peritoneal dialysis setting addresses a long-felt need for dialysis patients. Unfortunately, the presently used prior art transfer set 10 does not adequately address or facilitate enhanced disinfection techniques or devices.

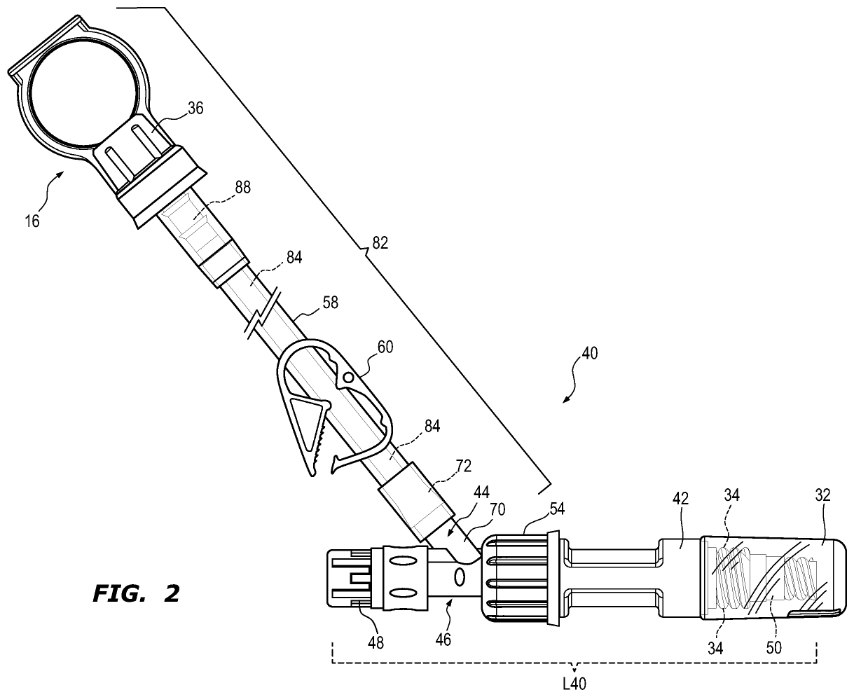

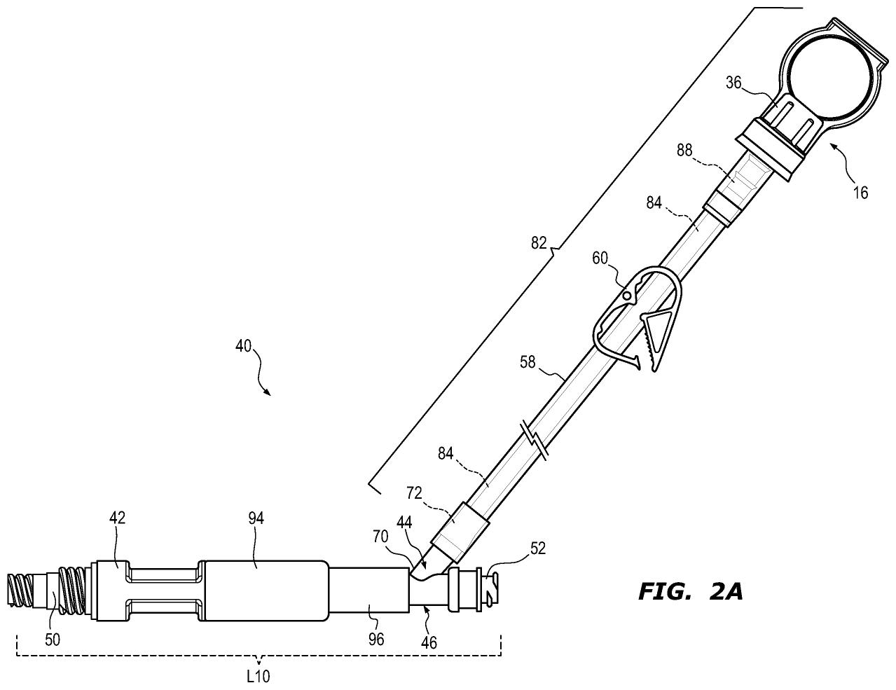

An exemplary transfer set 40 with an access port 44 of the present invention is depicted in FIG. 2 in a side elevational view. As depicted, transfer set 40 has a transfer set body 42, a Y-adapter 46 with the integral access port 44, a cap (such as a vented cap) 48, and a protective cap 32 and showing the luer connector 50 through the (transparent) protective cap 32.

FIG. 2A is a directionally reversed side elevational view of an alternative exemplary transfer set 40 with the vented cap 48 and protective cap 32 removed to reveal an exemplary male luer 52 and the luer connector 50, respectively.

The principal difference between the transfer set 40 depicted in FIG. 2 and the transfer set 40 depicted in FIG. 2A is that the transfer set 40 depicted in FIG. 2 may be originally manufactured in the form and configuration depicted, while the transfer set 40 depicted in FIG. 2A is constructed by modifying an existing, off-the-shelf prior art transfer set 10 (see FIGS. 1 and 1A) and adding off-the-shelf parts to create the form and configuration depicted. For purposes of this disclosure, the structure and function of the transfer set 40 depicted in FIG. 2 will be described first, and the description of the method of construction, structure, and function of the transfer set depicted in FIG. 2A will follow.

Turning now to FIG. 2, the transfer set 40 is multifunctional and further comprises a transfer set cap 54, coupler tubing 56, branch tubing 58, a pinch clamp 60, and a coupling assembly 16 with the ring cap 36. The transfer set body 42 (as shown in FIG. 3) is connected to the luer connector 50 (partially shown in FIG. 2, see FIG. 4B). The combination of the transfer set body 42 and the connected luer connector 50 defines a longitudinally aligned transfer set body assembly for the embodiment shown in FIG. 2. The luer connector 50 may be similar or identical to the core male luer 28 in that each may include the same threads and a hollow flange 30. Coupler tubing 56 is secured to the hollow flange 30. The luer connector 50 is threadedly secured to the transfer set body 42 such that the hollow flange 30, coupler tubing 56, and a portion of the luer connector 50 nest within the hollow of the transfer set body 42. The visible portion of the luer connector 50 is visible through and is being protected by a transparent protective cap 32. The protective cap 32, whether transparent or not, protects and maintains the sterility of the luer connector 50 while in place. In the embodiment shown, the protective cap 32 and the luer connector 50 have mating cap threads 34 that engage to secure the protective cap 32 to the luer connector 50; however, the female threads of the protective cap 32 are not shown so not to obscure the male threads (both male and female threads are mating cap threads 34) of the luer connector 50.

FIG. 3 is a side elevational view of the exemplary transfer set body 42 isolated from the exemplary transfer sets 40 of FIGS. 2 and 2A. Note that FIG. 3 also depicts hub body 20 of FIG. 1A with the pair of tines 22 and any bump stops 24 removed. The transfer set body 42 and the hub body 20 each have a female coupler such as female threads 62 (shown in phantom lines) and a male coupler such as male threads 64. The female threads 62 are for threaded engagement with the luer connector 50 and the core male luer 28, respectively. The male threads 64 of hub body 20 serve as functional structure of a rotational receiving coupler for the twisting open/close mechanism 18 that controls the flow of fluids through prior art transfer set 10. As noted above, this twisting open/close mechanism 18 comprises the hub body 20 with tines 22 (see FIG. 1A) and at least one bump stop 24 (see FIG. 1A), and a twist clamp 26 that engages the male threads 64 of hub body 20 in threaded engagement wherein twisting (rotating) the twist clamp 26 one direction until the bump stop 24 is encountered closes the prior art transfer set 10 by compressing the tines 22 to clamp shut the intermediate tubing 14 so that fluids are prevented from flowing through the prior art transfer set 10. Twisting (rotating) the twist clamp 26 the opposite direction from being closed threadedly opens the prior art transfer set 10 by releasing the tines 22 from clamping the intermediate tubing 14 so that fluids may freely flow through the prior art transfer set 10. The male threads 64 of the transfer set body 42 secure the Y-adapter 46 to the transfer set body 42 by threaded engagement of the transfer set cap 54.

FIGS. 4A-C depict a series of side elevational views showing an exemplary sub-assembly comprising a combination of the luer connector 50, the transfer set body 42, and coupler tubing 56. FIG. 4A depicts the transfer set body 42 as connected to luer connector 50. FIG. 4B depicts the coupler tubing 56 connected to the hollow flange 30 portion of the luer connector 50, the transfer set body 42 having been removed so not to obscure the coupler tubing 56. FIG. 4C is a vertical cross-section view of the connection of the coupler tubing 56 to the hollow flange 30 portion of the luer connector 50 disposed within a tubing lumen of the coupler tubing 56. The cross-section view reveals that the luer connector 50 also has a passageway 66 through which fluids flow when transfer set 40 is open.

The Y-adapter 46 is connected to the transfer set body 42 in any suitable manner. As shown in the exemplary embodiment of FIG. 2, the transfer set cap 54 has female threads 68 and is threaded onto the male threads 64 of the transfer set body 42.

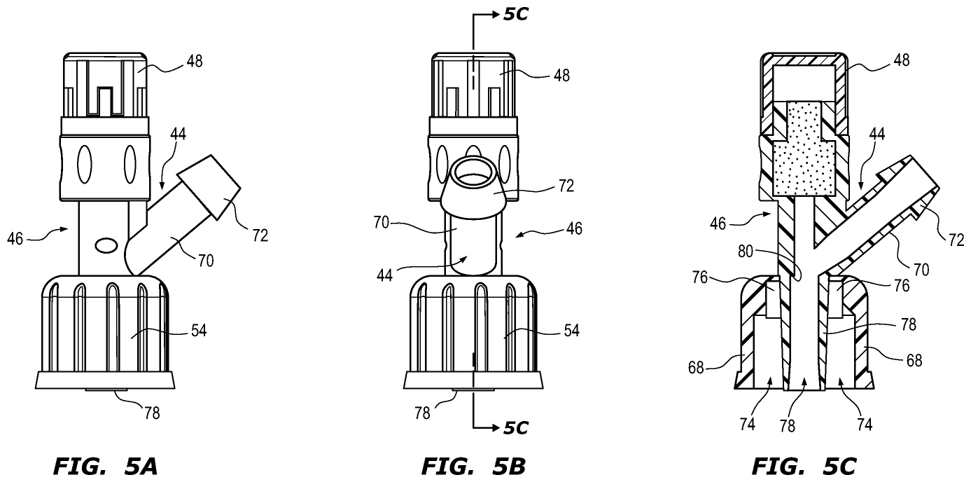

A series of side elevational views is depicted in FIGS. 5A-C showing the exemplary Y-adapter 46. FIG. 5A depicts the Y-adapter 46 which has the transfer set cap 54 and vented cap 48 attached and an integral side port 70 directed to the right. Side port 70 has a barbed end 72 for connecting and securing the branch tubing 58 to the side port 70. FIG. 5B depicts the Y-adapter 46 rotated 90° so that the barbed side port 70 is directed forward and upward. FIG. 5C depicts a sectional view along Section C-C of FIG. 5B showing the inner profile of the Y-adapter 46 and revealing an annular recess 74, an annular spacer 76 and a central hollow protrusion 78 in the transfer set cap 54. The annular recess 74 has female threads 68 that receives male threads 64 in threaded engagement so that the coupler tubing 56 and hollow flange 30 are disposed within the central hollow protrusion 78 and abut against a shoulder 80, completing a secure seal in the connection of the transfer set body 42 to the Y-adapter 46. However, those skilled in the art, armed with this disclosure, will understand that the connection of the Y-adapter 46 to the transfer set body 42 should not be limited to threaded engagement, but that connection may be accomplished in any suitable manner such as using adhesives, ultrasonic welding, press fitting, and the like.

The cap 48 (such as vented cap 48 in FIG. 2) protects the male luer 52 (shown best in FIG. 2A) from contamination and damage. However, those skilled in the art, armed with this disclosure, will understand that any suitable type of connector may be used and may be dependent on the type of device the Y-adapter 46 is to be connected. For example, there are many types of male luers that could be used, as well as female luers, threads, slot-and-post, and any other suitable connector.

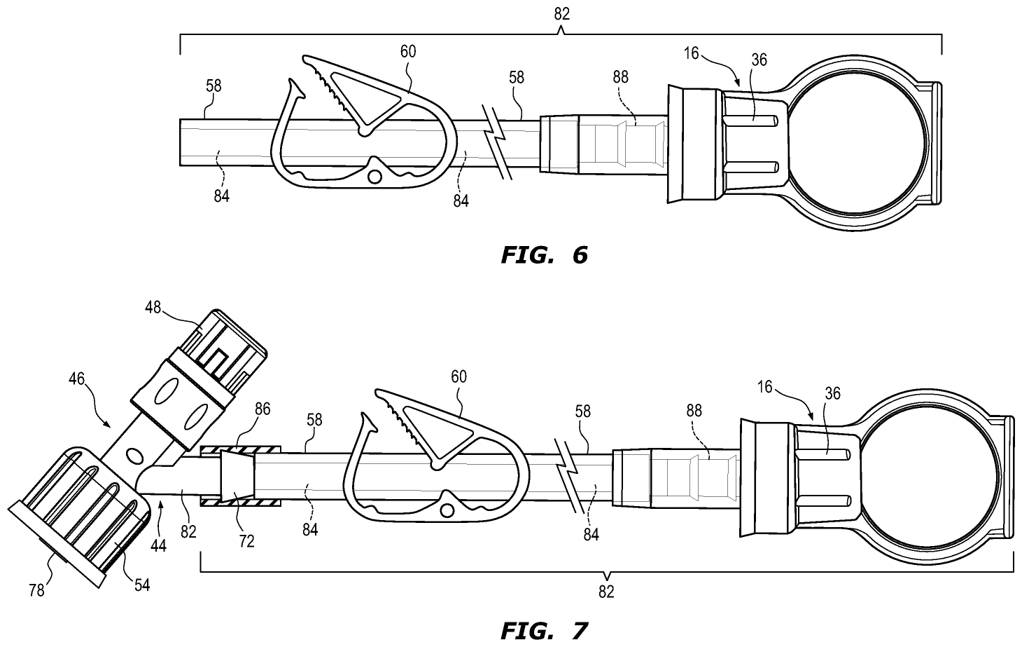

Transfer set 40 of FIG. 2 further comprises an extension sub-assembly 82 connected to side port 70 and connectable to a catheter or some other structure the permits the flow of fluids therethrough. The extension sub-assembly 82 comprises the branch tubing 58 with a lumen 84 defined by the interior wall of the branch tubing 58, a compression sleeve 86, pinch clamp 60, a barbed connector 88 shown in phantom lines, a luer (not visible in FIG. 6 or 7) that may have either male threads or female threads or any other suitable luer means for connecting the extension sub-assembly 82 to the catheter or any other fluid-flow-permitting structure, and a ring cap 36 to protect and maintain the sterility of the luer threads or other luer connection means. Use of ring cap 36 facilitates single-hand detachment/reattachment of the ring cap 36 from the transfer set 40.

The branch tubing 58 is stretched over and envelops the barbed end 72 of the side port 70 and secured by the compression sleeve 86 that overlays the portion of the branch tubing 58 that extends over the barbed end 72 and compresses that connection to enhance the securement and reduce any chance of disconnection.

Turning back to FIG. 2A, the alternative exemplary transfer set 40 depicted is constructed by modifying an existing, off-the-shelf prior art transfer set 10 (see FIGS. 1 and 1A) and adding off-the-shelf parts to create the form and configuration shown. Because this alternative transfer set 40 is also multifunctional and is constructed from parts of an existing prior art transfer set 10 and off-the-shelf parts, some the reference numbers used to describe the prior art transfer set 10 will be used, along with the reference numbers used to describe transfer set 40, so that it may be clearly understood the method of converting prior art transfer set 10 is converted into the alternative transfer set 40 of FIG. 2A.

To start, a prior art transfer set 10 of the type shown in FIGS. 1 and 1A, is obtained and dismantled by separating the intermediate tubing 14 from the transfer set assembly 12. Further, the transfer set assembly 12 may be separated into the core male luer 28 and the hub body 20. Tines 22 and any bump stops 24 are removed from hub body 20 (compare FIG. 1A to FIG. 3), preferably by precision cutting and preferably without sanding or filing that may create loose small particles or shavings. Such loose small particles or shavings may contaminate the sterile environment or inhibit proper sealing of contact surfaces. The removal of the tines 22 and bump stop(s) 24 converts the hub body 20 into the form and configuration of the transfer set body 42 of FIG. 3.

So that components remain secured together, a bonding solvent or adhesive may be applied to secure components from pulling, twisting, or rotational disengagement. For example, to secure the coupler tubing 56 to the hollow flange 30 a bonding solvent or adhesive may be applied to the inner wall of the coupler tubing 56 before the hollow flange 30 is inserted into the lumen of the coupler tubing 56. Making certain that coupler tubing 56 is fully seated against the abutment shoulder 90 further assures that the connection of coupler tubing 56 to the hollow flange 30 seals properly. Additionally, bonding solvent or adhesive may be applied to female threads 62 of the modified hub body 20 (now a transfer set body 42) and/or inward male threads 92 of the core male luer 28 (now luer connector 50) to secure the threaded engagement of the transfer set body 42 to the luer connector 50.

The transfer set body 42 as bonded with the luer connector 50 may be connected to the twist clamp 26 and secured by bonding solvent or adhesive in an open position that disables it from twisting, thereby creating a disabled twist clamp 94. As shown in FIG. 2A, an intermediate connector 96 is connected between the disabled twist clamp 94 and an off-the-shelf Y-adapter 46 that has a male luer 52, side port 70, and connection end (not visible) that connects to the intermediate connector 96. The combination of the transfer set body 42, the connected luer connector 50, the disabled twist clamp 94, and intermediate connector 96 defines a longitudinally aligned transfer set body assembly for the embodiment shown in FIG. 2A. The connection end of the longitudinal portion of Y-adapter 46 may be of any suitable type (such as threaded, press fit, luer fitting, bonded or adhered, and the like) so long as it securely connects the Y-adapter 46 to the intermediate connector 96.

The intermediate tubing 14 may be cut to a desired length and fitted with an off-the-shelf flow-control device such as pinch clamp 60, thereby becoming branch tubing 58. The flow-control device (pinch clamp 60) allows or prevents fluid flow through the branch tubing 58. A tubular compression sleeve 86 may also be included in converting the intermediate tubing 14 into branch tubing 58. The branch tubing 58 (previously, intermediate tubing 14) may then be secured to the side port 70 by inserting the barbed end 72 of the side port 70 into the lumen 84 of the now branch tubing 58 and advancing the compression sleeve 86 to augment the security of the connection of tubing 58 drawn over the barb. Also, an adhesive may be used on the barbed end 72 to adhere the side port 70 to the newly converted branch tubing 58.

Additionally, a conversion of a prior art transfer set 10 into a transfer set 40 (as depicted in FIG. 2) may be accomplished by modifying the hub body 20 by removing the tines 22 and any bump stops 24 to convert the hub body 20 into a transfer set body 42 as described above, and then attaching the Y-adapter 46 to the male threads 64 of the newly converted transfer set body 42 via a transfer set cap 54 having corresponding female threads 68 (see FIG. 5C). The transfer set cap 54 may be of a type that seals by compressing the annular spacer 76 against the central hollow protrusion 78 extending from the Y-adapter 46 as the male threads 64 of the newly converted transfer set body 42 tightened in threaded engagement with the female threads 68 of the transfer set cap 54; or alternatively, the transfer set cap 54 may be bonded or adhered to Y-adapter 46 using any suitable bonding or adhesive such as a bonding solvent, UV adhesive that is cured, and the like.

Although prior art transfer sets 10 and other types of transfer sets have been known for decades, they have not been multifunctional, allowing access for any elongate device into the flow of fluid through the transfer set or into any passageway within the transfer set or any tubular structure connected to the transfer set. Simply put, known transfer sets have not had access ports that would permit access without disconnecting the transfer set. Hence, introducing and inserting elongate members such as, for example, a guide wire, an endoscope, an angioscope, a hysteroscope, a gastroscope, a flexible telescope for colonoscopies, bronchoscope, a cystoscope, probes for illumination, probes for pinpoint medication or treatment delivery, probes for inflation gas delivery, fiber optics, and any of numerous other devices or instruments that are elongate and may be inserted into and/or retracted from a cavity of a patient's body and/or from within tubular structures outside a patient's body has not been possible with existing, non-multifunctional transfer sets. Any of these elongate members introduced upstream of the transfer set to operate downstream of the transfer set required length sufficient to cover the upstream distance to the transfer set, plus the length of the transfer set, and plus the distance downstream of the transfer set needed to operate as intended. Such extended length may cause additional cost, handling difficulties, and/or may jeopardize the effectiveness of the elongated instrument inserted.

Hence, a method for converting an existing transfer set 10 into a multifunctional transfer set 40 with an access port 44 may proceed as follows:
Select an existing transfer set 10 with a transfer set hub assembly 12, intermediate tubing 14, a coupling assembly 16, and a twist clamp 26, wherein the transfer set hub assembly 12 has a core male luer 28 and a hub body 20 with tines 22 and at least one bump stop 24. The core male luer 28 has a hollow flange 30.
Detach the intermediate tubing 14 from the transfer set hub assembly 12.
Detach the core male luer 28 from the hub body 20.
Detach the twist clamp 26 from the hub body 20.
Remove the tines 22 from the hub body 20, preferably by precision cutting and preferably without sanding or filing that may create loose small particles or shavings. Such loose small particles or shavings may contaminate the sterile environment or inhibit proper sealing of contact surfaces.
Remove any bump stop 24 from the hub body 20, again preferably by precision cutting. This converts the hub body 20 into a transfer set body 42.
Attach coupler tubing 56 to the hollow flange 30, thereby converting the core male luer 28 into a luer connector 50. This attachment may be secured from detrimental movement by any suitable means of securement, including applying bonding solvent and/or applying an adhesive.
Attach the luer connector 50 to the transfer set body 42, forming a transfer set body assembly. This attachment may be secured from detrimental movement by any suitable means of securement, including applying bonding solvent, applying an adhesive, press fitting, and/or ultrasonic welding.
Obtain a complementary Y-adapter 46 with a side port 70 and attach the Y-adapter 46 to the transfer set body assembly.
Positioning a flow-control device, such as pinch clamp 60, onto the intermediate tubing 14. Any suitable type of flow-control device may be used so long as it regulates allowing and preventing the flow of fluid through the intermediate tubing 14 into and out of the Y-adapter 46.
Attach the intermediate tubing 14 to the side port 70 of the Y-adapter 46. This attachment may be secured from detrimental movement by any suitable means of securement, including applying bonding solvent and/or applying an adhesive.

An additional method for converting an existing transfer set 10 into an alternative exemplary multifunctional transfer set 40 with an access port 44 may have the following alternative steps:
Attach twist clamp 26 to the transfer set body assembly and disable the twist clamp 26 from rotating with respect to the transfer set body assembly. This converts twist clamp 26 into a disabled twist clamp 94. This attachment may be secured from detrimental movement by any suitable means of securement, including applying bonding solvent, applying an adhesive, press fitting, and/or ultrasonic welding.
Connect the disabled twist clamp 94 to Y-adapter 46 with side port 70, This connection may be directly to the disabled twist clamp 94 and connecting the Y-adapter to the disabled twist clamp 94 or via an intermediate connector 96. This connection may be secured from detrimental movement by any suitable means of securement, including applying bonding solvent, applying an adhesive, press fitting, and/or ultrasonic welding.

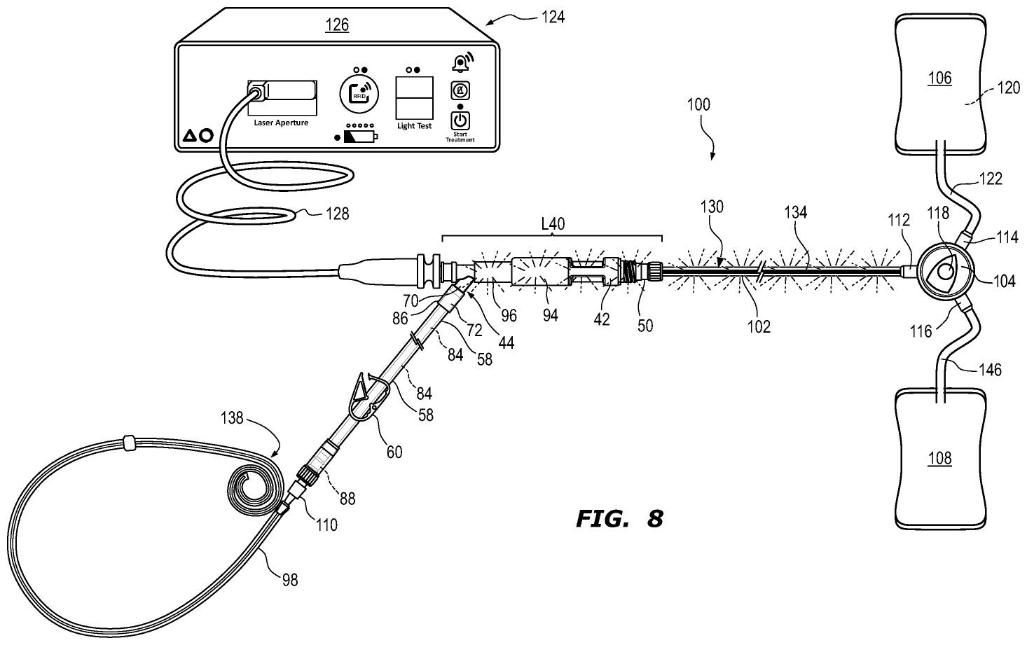

FIG. 8 is a perspective, schematic view of an exemplary peritoneal dialysis system 100 utilizing transfer set 40 with integral access port 44 of the type shown in FIG. 2A to facilitate the delivery of disinfecting/sterilizing light to a targeted area. The depiction is a basic representative embodiment of peritoneal dialysis systems 100. The use of this basic representation is not intended to be limiting of the scope of the present invention; rather, this disclosure contemplates and considers the use of the disclosed invention within more sophisticated peritoneal dialysis systems, known and yet to be developed, to be within the scope of the present invention. Armed with this disclosure and the disclosure of U.S. Pat. No. 11,229,728 issued Jan. 25, 2022, those skilled in the art will understand where, when, and how the delivery of non-ultraviolet electromagnetic radiation (referred to as EMR or light) may be used in more sophisticated peritoneal dialysis systems, known and yet to be developed.

As mentioned above, a few weeks before a patient starts peritoneal dialysis, a surgeon places a permanent, soft tube catheter into the patient's abdomen. In peritoneal dialysis, a specific solution, known as a dialysate, is introduced through the permanent, indwelling peritoneal dialysis catheter (PD catheter 98 in FIG. 8) in the lower abdominal cavity and when the dialysis cycle is completed the dialysate delivery is discontinued and the indwelling PD catheter 98 capped off so that the patient may move freely about.

The basic peritoneal dialysis system 100 (depicted in FIG. 8) comprises dialysis access through the peritoneum into a person's abdominal cavity via PD catheter 98, an external fluid extension line 102, a dialysate exchange switch 104, a dialysate supply bag 106, and a waste dialysate retrieval bag 108. An external coupling end 110 of PD catheter 98 is connected to the access port 44 of transfer set 40 and the luer connector 50 of transfer set 40 is connected to the fluid extension line 102. Fluid extension line 102 is connected to the dialysate exchange switch 104. The dialysate exchange switch 104 has an extension line portal 112, a dialysate inlet 114, a waste dialysate outlet 116 and an exchange selector 118 for selecting fluid flow paths. The dialysate supply bag 106 contains dialysate 120 (also referred to as dialysis solution 120) and is connected to the dialysate exchange switch 104 via a feed line 122 and the dialysate inlet 114, establishing a dialysate flow path (when the exchange selector 118 is moved to select dialysate flow) from the dialysate supply bag 106 into the feed line 122, through the dialysate exchange switch 104, into and through the fluid extension line 102 then the transfer set 40, to the PD catheter 98 for delivery into the patient's body.

Particularly with presently conducted at-home peritoneal dialysis treatments (though lesser so with institutional treatments), dialysis patients leave the transfer set 10 connected to the PD catheter 98 for as long as six to nine months capping off the transfer set 10, rather than the PD catheter 98, so that they may move about freely. They secure the external coupling end 110 of the PD catheter 98 and the connected transfer set 10 against their body using a wrapping, tape, or specially made belt under their clothing for comfort and so not to draw attention to the apparatus. As one might expect, this practice subjects these external components to possible contamination and infection vulnerability.

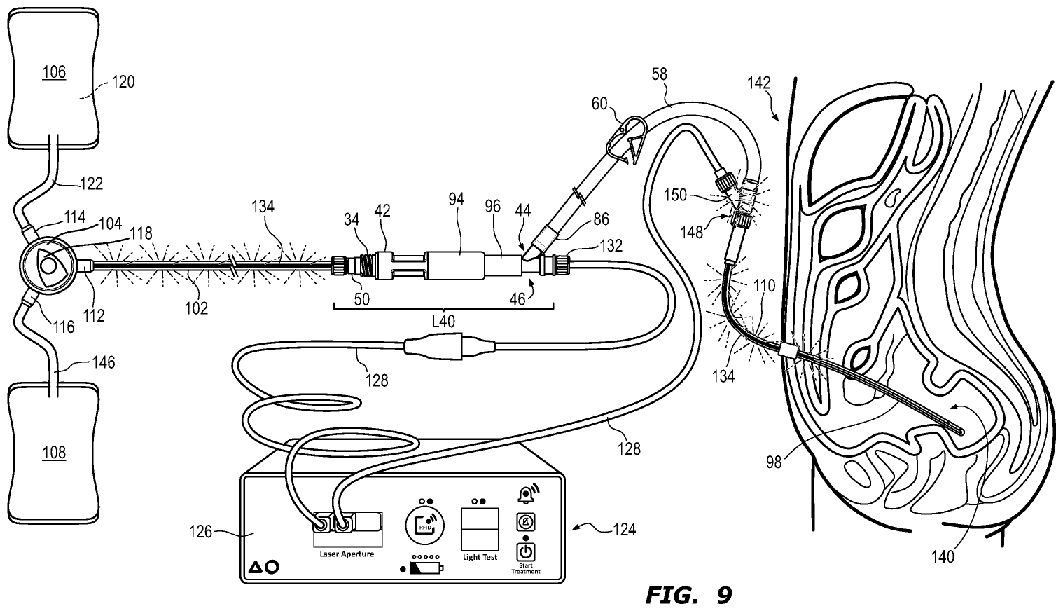

To address these types and other types of possible contamination and infection vulnerabilities the peritoneal dialysis system 100 may be enhanced by adding an EMR conduction system 124 to provide non-ultraviolet light disinfection and sterilization to the dialysis procedure prior to, during, and/or after a treatment. The EMR conduction system 124 comprises a light engine 126, a light transmission cord 128, and a light delivery element 130 (a portion of which is shown in FIG. 11). The light delivery element 130 comprises a connector assembly 132 having a SMA fiber connector 174 (best shown in FIG. 11) and a fiber optic 134 (see also FIGS. 9 and 11) This enhancement of the peritoneal dialysis system 100 may be part of a kit that includes the peritoneal dialysis system 100 and the EMR conduction system 124 (whether the EMR conduction system 124 is permanently connected to the peritoneal dialysis system 100 or removably insertable into the peritoneal dialysis system 100). Additionally, the EMR conduction system 124 may be retrofitted with an existing peritoneal dialysis system 100. As depicted, the fiber optic 134 of the EMR conduction system 124 has been introduced into fluid extension line 102 through the transfer set body 42 of transfer set 40 that facilitates the passage of the fiber optic 134 into the lumen of the fluid extension line 102 without impairing the free flow of fluid (e.g., dialysate 120 whether fresh or waste) into or out of and through the PD catheter 98.

An alternative kit of disposable components (at least components that are replaced more frequently than the PD catheter 98 and perhaps less frequently than the peritoneal dialysis system 100) may comprise transfer set 40 with integral access port 44 and light delivery elements 130 of various lengths. Such kits enable pairing the light delivery element 130 which has a fiber optic 134 of a predetermined longitudinal length with a transfer set 40 (having a longitudinal length L40 that is considerably less than the longitudinal length L10 of known transfer sets 10). For example, referring to FIG. 8, the length of fiber optic 134 extends through transfer set 40 into and through fluid extension line 102 up to extension line portal 112, and radial emission of disinfecting/sterilizing light from one or more radial emission segments along fiber optic 134 may target anywhere along the length of the fiber optic 134 from within the transfer set 40 to the extension line portal 112. Specifically, FIG. 8 depicts the radial emission of disinfecting/sterilizing light both within the transfer set 40 and fluid extension line 102. However, and by way of example, those skilled in the art, armed with this disclosure, will understand that a longer fiber optic 134 could extend into the dialysate exchange switch 104 and could emit radially into the dialysate exchange switch 104 and/or there could be radial emission within the transfer set 40 only with length of fiber optic 134 being longitudinal length L40. In short, the fiber optic 134 may be any suitable length dependent upon the equipment used and the desired targets for disinfection/sterilization.

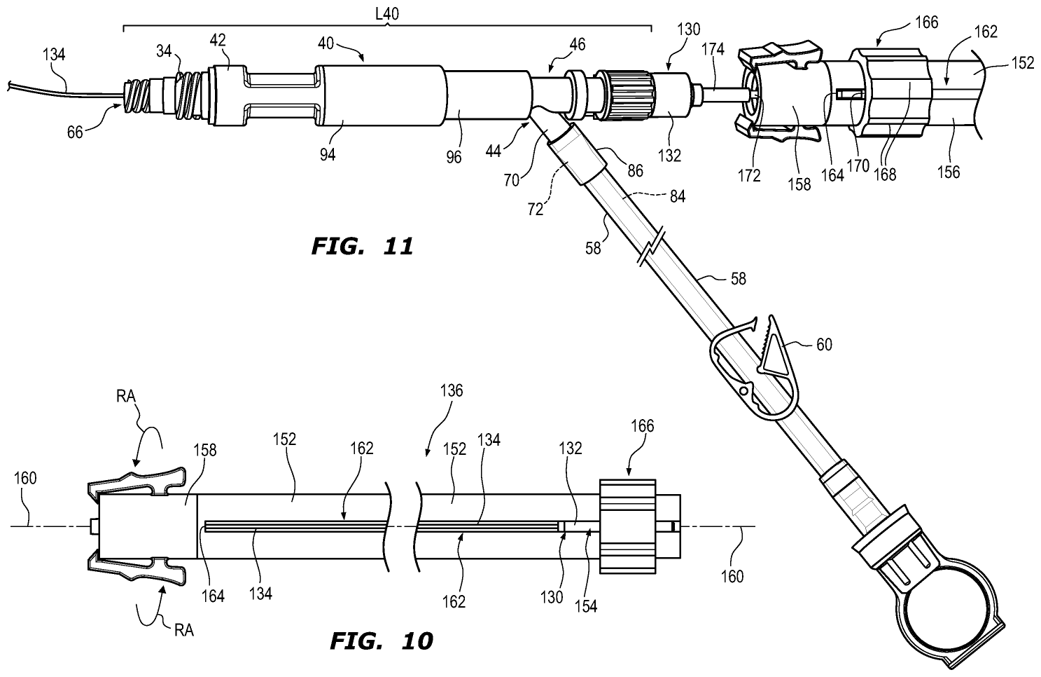

Also, using the multifunctional transfer set 40 offers significant advancements over the long-used transfer set 10. In general, multifunctional transfer set 40 has enhanced capabilities of receiving an elongate member or device insertably/retractably therethrough and allowing fluid to flow therethrough without being impeded unduly, and once connected there is no need to disconnect transfer set 40 to enable the insertion/retraction of a light delivery element 130 or any different right-sized elongate member or device or to enable the injection of a medication or the like. Also, the length of the fiber optic 134 and the length the introducer assembly 136 (not shown in FIG. 8, discussed below regarding FIGS. 10 and 11) is reduced because longitudinal length L40 (comprised of the transfer set body assembly (including but not limited to, embodiments shown in FIGS. 2, 2A, 8, 9, and 11) as connected to the longitudinal portion of Y-adapter 46) is less than L10.

Although, each of the multifunctional transfer sets 40 depicted in FIGS. 8, 9, and 11 are of the embodiment described regarding FIG. 2A, wherein the transfer set 40 is constructed by modifying an existing prior art transfer set 10 (see FIGS. 1 and 1A) and adding off-the shelf parts to create the form and configuration depicted. This alternative, exemplary embodiment is identified by the configuration having a disabled twist clamp 94 and intermediate connector 96. The retention of the disabled twist clamp 94 and intermediate connector 96 makes the overall longitudinal length L40 for this embodiment greater than the exemplary embodiment of transfer set 40 depicted in FIG. 2, wherein the transfer set cap 54 is threadedly connected directly to the male threads 64 of transfer set body 42. The embodiment of transfer set 40 depicted in FIG. 2 has an overall longitudinal length L40 considerably less than longitudinal length L40 because the twist clamp 26 and intermediate connector 96 have been eliminated either my original manufacture or by construction by modifying an existing prior art transfer set 10.

FIGS. 8 and 9 show similar, but different, peritoneal dialysis configurations of components during the dialysate 120 infusion phase of a dialysis exchange. The exchange selector 118 of the dialysate exchange switch 104 is directed towards the feed line 122 for the dialysate supply bag 106. This indicates that fresh dialysate 120 is flowing from the dialysate supply bag 106 through feed line 122 and dialysate exchange switch 104 into fluid extension line 102 (that is receiving disinfecting, sterilizing, or therapeutic healing light by radial emission from fiber optic 134). The light-treated dialysate 120 then flows from the fluid extension line 102 into transfer set 40 through the access port 44 into and through branch tubing 58 (note that pinch clamp 60 is open, allowing dialysate 120 to flow freely) and into the attached PD catheter 98 (as depicted in FIG. 8, PD catheter 98 is of the Tenckhoff variety, having a Tenckhoff coil 138) for discharge into the peritoneal cavity 140 of the patient 142 (shown in FIG. 9).

The peritoneal cavity 140 is surrounded by the peritoneal membrane also known as the peritoneal lining 144. Peritoneal lining 144 contains many blood vessels. Dialysate 120 draws extra fluid, chemicals, and waste out of those blood vessels and through the peritoneal lining 144. Peritoneal lining 144 acts as a filter. The dialysate 120 is left in place for a prescribed period up to several hours while dialysis occurs. Then the old, waste-laden solution (also known as waste dialysate 120) is allowed to drain out through the PD catheter 98 for disposal. Fresh, clean solution (dialysate 120) is immediately delivered in, filling in the space again. This process of exchanging waste dialysate 120 with fresh dialysate 120 is called an exchange.

Delivering disinfecting, sterilizing, and/or therapeutic healing light by radial emission from fiber optic 134 may be done during different periods and in different ways than are shown in FIG. 8. FIG. 8 depicts one example of light delivery during a period of dialysate 120 infusion, while FIG. 9 depicts two examples of light delivery during dialysate 120 infusion. Using the same configurations of components as depicted in FIGS. 8 and 9 or with slight modifications, various other ways of delivering light and during different periods may be described without necessitating additional drawings, while still being understood by those skilled in the art. Therefore, in the interest of brevity and avoiding numerous drawings with only slight changes, this disclosure will describe representative examples of various other ways of delivering light and during different periods.

Referring to FIGS. 8 and 9, by changing the exchange selector 118 of the dialysate exchange switch 104 so that it is directed towards the waste drain line 146 for draining the waste dialysate 120, either by gravity or by a cycler, into the waste dialysate retrieval bag 108 for disposal. This change in the position of the exchange selector 118 indicates that waste dialysate 120 is flowing from peritoneal cavity 140 of the patient 142 through the PD catheter 98 into and through branch tubing 58 (pinch clamp 60 being open) and the access port 44 of transfer set 40 flowing into fluid extension line 102 (that is receiving disinfecting or sterilizing light by radial emission from fiber optic 134). The light-treated waste dialysate 120 then flows from the fluid extension line 102 into dialysate exchange switch 104 and is directed into waste drain line 146 (making the dialysate exchange switch 104 and drain line 146 less likely to be contaminated by infections being drawn the patient 142) into the waste dialysate retrieval bag 108 that may be removed for disposal after the exchange is completed.

By changing the exchange selector 118 of the dialysate exchange switch 104 so that it selects neither the dialysate supply bag 106 nor the waste dialysate retrieval bag 108, the flow of dialysate 120 through the fluid extension line 102 is stopped. In this configuration, two situations are presented. The first situation would be pre-treatment, in other words, the configuration of components is newly configured as depicted in FIG. 8 or 9, but no fluid has yet gone through the components, other than possibly the indwelling PD catheter 98. In this situation, disinfecting or sterilizing light may be emitted by radial emission from fiber optic 134 as a preventative measure to reduce or eliminate any infectious agents that have been introduced during the assembling of the components even though those components are typically sterile before handling. The second situation would be after a dialysis session is complete and dialysate 120 flow has been stopped at the dialysate exchange switch 104 and/or at the pinch clamp 60. In this situation, disinfecting or sterilizing light may be emitted by radial emission from fiber optic 134 to reduce or eliminate any infectious agents that remains in the components as residue within the fluid extension line 122 or within the transfer set 40.

FIG. 9 differs from FIG. 8 in that it depicts an exemplary configuration with two light delivery elements 130 showing radial emission of light from two separate fiber optics 134 disposed in two distinct locations, one that provides disinfecting and/or sterilizing light by radial emission disposed within the fluid extension line 102 and the other provides disinfecting and/or sterilizing light by radial emission disposed within the PD catheter 98 and its external coupling end 110 and a Y-adapter 148 having a light coupling port 150. As depicted in FIG. 9, the PD catheter 98 is different from the variety of PD catheter 98 shown in FIG. 8 and the Y-adapter 148 with the light coupling port 150 is positioned inline between the branch tubing 58 of transfer set 40 and the external coupling end 110 of PD catheter 98. The length of the light emitting segment of the fiber optic 134 extends through the light coupling port 150 of Y-adapter 148, the external coupling end 110 of PD catheter 98, and into a portion of the PD catheter 98 extending into the patient's 142 peritoneal cavity 140. The fiber optics 134 shown may be of the same length or different lengths and may emit the same or different intensities and wavelengths of light simultaneously, alternatingly, alternatively, and any combination thereof.

The configurations depicted in FIGS. 8 and 9 are exemplary embodiments of possible peritoneal dialysis configurations that demonstrate the versatility of the component parts, particularly transfer sets 40, and is not intended to be limiting of the scope of the invention herein disclosed. Those skilled in the art, armed with this disclosure will understand and be able to implement transfer sets 40 with various forms of disinfecting/sterilizing/therapeutic healing light delivery using fiber optics 134 for radial light emission.

Turning to FIGS. 10 and 11, an exemplary introducer assembly 136 is depicted. In FIG. 10 the exemplary introducer assembly 136 is shown in a ready mode, fully assembled and ready for use, the ready mode being a configuration of the introducer assembly 136 during shipping and/or storage typically. The exemplary introducer assembly 136 comprises two principal components, a disposable guide tube 152 and an elongate member assembly 154.

The guide tube 152 may be disposable and may be constructed in dimensions ergonomically complementary to its intended use and/or target users and may be made of any suitable material that is cost-effective and complementary to its use. For example, and not limited to this example, the guide tube 152 may be constructed of lightweight materials with a larger diameter and larger movable parts balanced to fit comfortably in a person's 142 hand that may have limited dexterity, poor vision, and/or some other limiting characteristic(s) that may otherwise inhibit proper use of a less well-sized or smaller guide tube 152. Furthermore, the elongate member assembly 154, as housed within the guide tube 152 while in a ready mode, may be advanced within the guide tube 152 to an introduced mode wherein the fiber optic 134 of light delivery element 130 is introduced into a desired disposition such as within a tubular structure (e.g., a bore, tubing, a conduit, a catheter, transfer set, extension line, and the like). Because the fiber optics 134 of light delivery elements 130 are made to target light emission within various systems and to various locations, the guide tube 152 may require larger/smaller diameters, longer/shorter lengths, and larger/smaller other components to accommodate the selected fiber optic 134 of each light delivery element 130 to be used.

Additionally, there are numerous medical uses requiring introduction and/or retraction of medical instruments into a patient's body 142 or into a medical device or component. Again, by way of example and not to be limiting, it may be beneficial and cost-effective to use the guide tube 152 to insert and/or retract a medically related elongate member, such as a fiber optic 134 that may require one time or repeated insertion and/or retraction. It is contemplated that, armed with the disclosure herein, persons of ordinary skill in the art could fashion guide tubes 152, disposable or not, to advance and/or retract such devices or instruments without undue experimentation.

For conciseness, the introducer assembly 136 described herein is intended to be a representative, exemplary embodiment. The representative, exemplary embodiment of the introducer assembly 136 described herein has medical context by introducing a fiber optic 134 into a tubular-receiving structure such as a catheter, a transfer set, and/or an adapter and serves as a representative description of other similar embodiments.

To simplify the description of the introducer assembly 136, FIGS. 10 and 11 depict a representative example of all introducer assemblies 136 contemplated herein comprising a guide tube 152 and an elongate member assembly 154. Guide tube 152 may be disposable or not but is particularly suitable for disposability because it may be removed from the elongate member assembly 154. Guide tube 152 comprises a proximal body 156 and a distal portion 158 and has a longitudinal axis 160 about which the distal portion 158 may be rotated relative to the proximal body 156 as depicted by rotation arrows RA in FIG. 10. Proximal body 156 has a longitudinal slide slot 162 with a stop 164. As mentioned above, the dimensions and the materials with which the guide tube 152 is made may be determined to accommodate whatever type of fiber optic 134 is selected to be used.

The component parts of introducer assembly 136, in addition to guide tube 152 described above, comprise a slide collar 166 having a grip 168, a slide post 170, a centered capture structure having a centered annular cylinder (not shown, centered about the longitudinal axis 160), a proximal connector assembly 132 configured to nest within the centered annular cylinder, and a distal connector assembly 172 configured to nest rotatably with the distal portion 158. Additionally, an at least partially annular snap-fit joint (not visible) facilitates the rotation of a distal portion 158 relative to a proximal body 156 of the guide tube 152.

The centered capture structure need not be annular or circular so long as it grasps or captures the proximal connector assembly 132 in a nesting engagement so that the elongate member assembly 154 advances longitudinally along the longitudinal axis 160. For example, the centered capture structure may be any of a number of structures that captures and secures the proximal connector assembly 132 during advancement of the slide collar 166 such as a partial cylinder, a hollow frustoconical structure (whether annular or partially annular), a rectangular tube or partially rectangular tube (if the transverse profile of the proximal connector assembly 132 fits therewithin and is captured), a non-circular tube or partial non-circular tube (if the transverse profile of the proximal connector assembly 132 fits therewithin and is captured), or any other capturing structure that secures and advances the proximal connector assembly 132 during the advancement of the sliding collar 166 and may easily release the proximal connector assembly 132 when guide tube 152 is removed from the elongate member assembly 154. Because the transverse profile of the proximal connector assembly 132 may have almost any shape, those skilled in the art, armed with this disclosure, may easily fashion a centered capture structure without undue experimentation.

FIG. 10 depicts various component parts of the introducer assembly 136 while disposed in the ready mode. The ready mode is the configuration that would be most suitable for shipment and storage. In fact, the introducer assembly 136 may be pre-sterilized and packaged to maintain sterility during shipment and storage. In the ready mode the slide collar 166 is slidably connected to the guide tube 152 such that the grip 168 is accessible to the user while the user holds the guide tube 152, the slide post 170 slidably engages the slide slot 162, and the centered annular cylinder is centered about the longitudinal axis 160 and may travel along the longitudinal axis 160 as the grip 168 is advanced from the ready mode to the introduced mode.

The proximal connector assembly 132 nests within the centered annular cylinder and holds the elongate member; namely, a fiber optic 134 in the depicted exemplary embodiment, to extend along the longitudinal axis 160. For connecting proximal connector assembly 132 to the fiber optic 134, an SMA fiber connector 174 (SubMiniature A connector) may be used to securely grasp or hold the proximal end of the fiber optic 134 so that light transfers from a light source, such as light engine 126, to the fiber optic 134 for axial propagation along the fiber optic 134.

Stop 164 prevents distal portion 158 from advancing too far and/or from over rotating and damaging the distal connector assembly 172. The combination of advancing the slide collar 166 and rotating the distal portion 158 to couple together the proximal connector assembly 132 and the distal connector assembly 172 moves the introducer assembly 136 from the ready mode where the fiber optic 134 is housed within the guide tube 152 to the introduced mode where the fiber optic 134 is advanced fully through and extends from the distal connector assembly 172.

FIG. 11 depicts a perspective view of an exemplary transfer set 40 with integral access port 44 showing the fiber optic 134 as introduced into and through the transfer set body 42 prior to the introducer assembly 136 being disengaged and withdrawn. As the introducer assembly 136 is withdrawn, it enters its detached mode (as depicted in FIG. 11). In the detached mode the introducer assembly 136 may be disposed of or, depending on the introducer assembly 136, it may be reattached so that the fiber optic 134 may be retracted for disposal or re-introduction after sterilizing.

As shown in FIG. 11, fiber optic 134 having been introduced into and through the transfer set body 42 extends out of passageway 66 of luer connector 50. The length of the fiber optic 134 corresponds to the length needed to reach whatever location or locations targeted for receiving radial emission of light. For example, if the transfer set 40 is to be connected to an extension line 102 (as shown in FIG. 8), the length of the fiber optic 134 may be longitudinal length L40 plus the length of the extension line 102, or if it is desired to deliver radially emitted light into the dialysate selector switch 104 connected to extension line 102, the length may be a bit longer so that the fiber optic 134 extends into the dialysate selector switch 104. However, the fiber optic 134 need only have a longitudinal length L40 if the delivery of radially emitted light is only needed within the transfer set 40. Simply put, the length of the fiber optic 134 may be custom made to fit the needed light delivery based on the component parts through which the fiber optic 134 extends, or various stands lengths may be made to fit known configurations of component parts.

Various exemplary embodiments disclosed herein describe configurations of component parts and arrangements of those component parts. The exemplary embodiments of the transfer sets 40 disclosed herein are extremely flexible and conducive to being combined with a wide range of known component parts or devices, making the transfer sets 40 of this disclosure ideal companions for kit distribution and complementary use platforms. For example, kits that include a transfer set 40 may be matched compatibly with numerous components, parts, and fittings. Implementing transfer sets 40 into the delivery of medical services and treatments will likely enhance overall performance, improve efficiency and efficaciousness, reduce costs, minimize errors, prevent/reduce/eliminate infections, and simplify assembly of parts by providing pre-packaged kits that comprise compatibly matching companion parts.

By way of example, and using peritoneal dialysis as a representative environment, utilizing non-ultraviolet light emitted radially from a fiber optic 134 can prevent infections (light targeting contamination-vulnerable locations prior to a dialysis exchange), reduce infection (light applied at first indications of infection before, during, or after an exchange), or eliminate infections (light applied at intensities and for dosing durations before, during, or after an exchange). A pre-packaged kit may contain a fiber optic 134 and a compatibly matching companion transfer set 40 where fittings match so that connections are secure and where the intended use is predetermined so that, for example, the fiber optic 134 has a diameter and the transfer set 40 has a through-lumen or passageway to receive the fiber optic 134 so that any fluid flow through the transfer set 40 and any tubular structure connected thereto (e.g., PD catheter 98, extension line 102, branch tubing 58, Y-adapter 148 with light coupling port 150 and the like) will not be occluded or otherwise impeded. Further the fiber optic 134 has a length that compatibly matches with the companion transfer set 40 to extend a desired length that takes the longitudinal length L40 of the transfer set 40 into account assuring that the reach of the fiber optic 134 is proper and that any radial emission segments of the fiber optic 134 are positioned within the transfer set 40 and/or any tubular structure connected thereto to emit the disinfecting/sterilizing light to pre-determined locations for effective treatments.

Another exemplary kit may further comprise an introducer assembly 136 that accommodates the size and length of the fiber optic 134.

Yet another exemplary kit may further comprise an extension line 102 that accommodates the size and length of the fiber optic 134 and is connectable securely to the transfer set 40.

Another exemplary kit may further comprise a PD catheter 98 that accommodates the size and length of the fiber optic 134 and is connectable securely to the transfer set 40.

Of course, there are many other configurations and structural arrangements that may benefit from the use of one or more transfer sets 40. Consequently, the various embodiments and configurations of transfer set 40 make the invention disclosed herein extremely versatile, retrofittable, and treatment enhancing.

Those skilled in the art will appreciate that the present embodiments are exemplary and should not be limited to the embodiments shown and described.

The present invention may be embodied in other specific forms without departing from its structures, methods, or other essential characteristics as broadly described herein and claimed hereinafter. The described embodiments and configurations are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A multifunctional transfer set having enhanced capability to receive an elongate member insertably/retractably therethrough and allowing fluid to flow therethrough, the multifunctional transfer set comprising:
a transfer set body assembly having a transfer set body and a luer connector, the transfer set body assembly having a passageway and a longitudinal axis each extending through the transfer set body and the luer connector;
a Y-adapter with an access port, the Y-adapter being connected to the transfer set body assembly such that the longitudinal axis extends through a longitudinal portion of the Y-adapter and not through the access port, the connected transfer set body assembly and longitudinal portion of the Y-adapter defines a longitudinal length for the multifunctional transfer set; and,
an extension sub-assembly connected to the access port, the extension sub-assembly comprises branch tubing, a flow-control device, and a coupling assembly with a ring cap, the extension sub-assembly allows fluid to flow therethrough into and out of the transfer set body assembly if the flow-control device is open and prevents fluid from flowing therethrough if the flow-control device is closed.

2. The multifunctional transfer set of claim 1, wherein the transfer set body assembly further comprises a disabled twist clamp connected to the transfer set body.

3. The multifunctional transfer set of claim 2, wherein the transfer set body assembly further comprises an intermediate connector connected to and disposed between the disabled twist clamp and the Y-adapter.

4. The multifunctional transfer set of claim 3, wherein the passageway and the longitudinal axis extending through the transfer set body and the luer connector also extend through the disabled twist clamp and the intermediate connector.

5. The multifunctional transfer set of claim 4, wherein the passageway insertably/retractably receives the elongate member therethrough.

6. The multifunctional transfer set of claim 1, wherein the passageway insertably/retractably receives the elongate member therethrough.

7. The multifunctional transfer set of claim 6, wherein the multifunctional transfer set facilitates the delivery of disinfecting/sterilizing light for preventing, reducing, and/or eliminating infectious agents within a dialysis system comprising an extension line, dialysate bags, and a dialysis catheter, wherein the extension line is connectable to and intermediate of the dialysate bags and the luer connector and the dialysis catheter is connectable to the coupling assembly of the extension sub-assembly, such that dialysate flowing between the dialysate bags and the dialysis catheter passes through the extension line, at least a portion of the longitudinal portion of the Y-adapter, the access port, the branch tubing, and the dialysis catheter;
wherein the elongate member comprises a connector assembly and a fiber optic, the connector assembly is connectable to the longitudinal portion of the Y-adapter such that the fiber optic extends into the longitudinal portion of the Y-adapter, the fiber optic for receiving disinfecting/sterilizing light and delivering disinfecting/sterilizing light to prevent, reduce, and/or eliminate infectious agents.

8. The multifunctional transfer set of claim 7, wherein the fiber optic has a length that extends into the extension line and is disposed to deliver disinfecting/sterilizing light by emission within the extension line.

9. The multifunctional transfer set of claim 1, wherein the flow control device is a pinch clamp disposed on the branch tubing.

10. The multifunctional transfer set of claim 1, wherein the multifunctional transfer set is packaged within a pre-packaged kit along with the elongate member, contents of the pre-packaged kit being sterilized and compatibly matched in size and structure to operate together with the elongate member to accomplish a designed purpose, the elongate member being selected from the group of elongate members consisting of a guide wire, an endoscope, an angioscope, a hysteroscope, a gastroscope, a flexible telescope for colonoscopies, bronchoscope, a cystoscope, probes for illumination, probes for pinpoint medication or treatment delivery, probes for inflation gas delivery, and fiber optics, wherein dimensions of the multifunctional transfer set compatibly match the elongate member selected such that a maximum diameter of the elongate member is less than a minimum diameter of the multifunctional transfer set.

11. The multifunctional transfer set of claim 10, wherein the elongate member is a fiber optic.

12. The multifunctional transfer set of claim 11, wherein the contents of the pre-packaged kit further comprises a introducer assembly, the introducer assembly disposed in a ready mode and having a length to receive and hold the fiber optic in position for introduction into the multifunctional transfer set.

13. The multifunctional transfer set of claim 11, wherein the contents of the pre-packaged kit further comprises an fluid extension line for connection to the multifunctional transfer set, the fiber optic having a length to extend through the multifunctional transfer set and into the fluid extension line.

14. A method of converting an existing transfer set into a multifunctional transfer set with an access port, the existing transfer set having a transfer set hub assembly, intermediate tubing, a coupling assembly, and a twist clamp, the transfer set hub assembly having a core male luer and a hub body with tines and at least one bump stop, the core male luer having a hollow flange, the method of converting the existing transfer set into a multifunctional transfer set with an access port comprises the steps of:
disassembling the existing transfer set comprising the steps of:
detaching the intermediate tubing from the transfer set hub assembly;
detaching the core male luer from the hub body; and
detaching the twist clamp from the hub body;
removing the tines from the hub body;
removing any bump stop from the hub body, thereby converting the hub body into a transfer set body;
attaching a coupler tubing to the hollow flange, thereby converting the core male luer into a luer connector;
attaching the luer connector to the transfer set body forming a transfer set body assembly;
obtaining a Y-adapter having side port and attaching the Y-adapter to the transfer set body assembly;
positioning a flow-control device onto the intermediate tubing, the flow-control device for allowing and preventing the flow of fluid through the intermediate tubing into and out of the Y-adapter; and
attaching the intermediate tubing to the side port of the Y-adapter.

15. The method of converting an existing transfer set into a multifunctional transfer set 14, wherein the steps of removing the tines from the hub body and removing any bump stop from the hub body are performed by precision cutting.

16. The method of converting an existing transfer set into a multifunctional transfer set 14, wherein the steps of attaching a coupler tubing to the hollow flange and attaching the intermediate tubing to the side port of the Y-adapter further comprises securing the attachment by selecting each securing step from the group consisting of applying a bonding solvent and applying an adhesive.

17. The method of converting an existing transfer set into a multifunctional transfer set 14, wherein the steps of attaching the luer connector to the transfer set body and attaching the Y-adapter to the transfer set body assembly further comprises securing the attachment by selecting each securing step from the group consisting of applying a bonding solvent, applying an adhesive, press fitting, and ultrasonic welding.

18. A method of converting an existing transfer set into a multifunctional transfer set with an access port, the existing transfer set having a transfer set hub assembly, intermediate tubing, a coupling assembly, and a twist clamp, the transfer set hub assembly having a core male luer and a hub body with tines and at least one bump stop, the core male luer having a hollow flange, the method of converting the existing transfer set into a multifunctional transfer set with an access port comprises the steps of:
disassembling the existing transfer set comprising the steps of:
detaching the intermediate tubing from the transfer set assembly;
detaching the core male luer from the hub body; and
detaching the twist clamp from the hub body;
removing the tines from the hub body;
removing any bump stop from the hub body, thereby converting the hub body into a transfer set body;
attaching a coupler tubing to the hollow flange, thereby converting the core male luer into a luer connector;
attaching the luer connector to the transfer set body forming a transfer set body assembly;
attaching the twist clamp to the transfer set body assembly and disabling the twist clamp from rotating with respect to the transfer set body assembly, thereby converting the twist clamp into a disabled twist clamp;
obtaining a Y-adapter having side port and connecting the Y-adapter to the disabled twist clamp;
attaching the intermediate tubing to the side port of the Y-adapter.

19. The method of converting an existing transfer set into a multifunctional transfer set 18, wherein the steps of attaching a coupler tubing to the hollow flange and attaching the intermediate tubing to the side port of the Y-adapter further comprises securing the attachment by selecting each securing step from the group consisting of applying a bonding solvent and applying an adhesive.

20. The method of converting an existing transfer set into a multifunctional transfer set 14, wherein the steps of attaching the luer connector to the transfer set body and connecting the Y-adapter to the transfer set body assembly further comprises securing the attachment by selecting each securing step from the group consisting of applying a bonding solvent, applying an adhesive, press fitting, and ultrasonic welding.
